(19) United States
(12) Patent Application Publication
(10) Pub. No.: US 2024/0350062 A1
(43) Pub. Date: Oct. 24, 2024

(54) ELECTRODE CONNECTOR WITH MOTION ARTIFACT DAMPENER

(71) Applicant: KPR U.S., LLC, Mansfield, MA (US)

(72) Inventors: Alexandra Mason, West Springfield, CA (US); Hannah Sugrue, Ludlow, MA (US); Nghia Nguyen, Chicopee, MA (US); Erick Garstka, Westfield, MA (US); Jacob Ameiss, Hazelwood, MO (US); Melvin Finke, Deland, FL (US)

(21) Appl. No.: 18/641,038

(22) Filed: Apr. 19, 2024

Related U.S. Application Data
(60) Provisional application No. 63/497,334, filed on Apr. 20, 2023.

(51) Int. Cl. A61B 5/274; A61B 5/28

(52) U.S. Cl. CPC A61B 5/274; A61B 5/28; A61B 2562/12

(57) ABSTRACT

A biomedical connector includes a housing defining an interior space and an opening dimensioned to receive an electrode at least partially into the interior space. A lever is movably attached to the housing and biased toward an engagement position for contacting the electrode when the electrode is received in the opening in the housing to retain the biomedical connector to the electrode. A dampener is disposed in the interior space of the housing and configured to engage the electrode when the electrode is received in the opening in the housing. The dampener is configured to limit movement of the biomedical connector relative to the electrode to reduce motion artifact in a biomedical signal received by the biomedical connector.

### CROSS-REFERENCE TO RELATED APPLICATION

This application claims priority to U.S. Provisional Patent Application Ser. No. 63/497,334, filed Apr. 20, 2023, and which is hereby incorporated by reference in its entirety.

### FIELD

The present invention generally relates to biomedical electrodes, and in particular, to an electrode connector for attaching a lead wire to an electrocardiogram (ECG) electrode placed on a patient's body, the electrode connector having a motion artifact dampener.

### BACKGROUND

When a patient requires monitoring for observation, treatment, or a combination of both, such as in a medical environment, e.g., a hospital, nursing home, or assisted living facility, the patient's vital signs and other health indicators may be monitored in order to continually and accurately assess the patient's well-being. One such vital sign is the monitoring of the heart via an electrocardiogram, which may be commonly referred to as an EKG and/or ECG.

Electrocardiograph (ECG) monitors are widely used to obtain medical (i.e. biopotential) signals containing information indicative of the electrical activity associated with the heart and pulmonary system. To obtain medical signals, ECG electrodes are applied to the skin of a patient in various locations. The electrodes, after being positioned on the patient, connect to an ECG monitor by a set of ECG lead wires. The distal end of the ECG lead wire, or portion closest to the patient, may include a connector which is adapted to operably connect to the electrode to receive medical signals from the body. The proximal end of the ECG lead set is operably coupled to the ECG monitor and supplies the medical signals received from the body to the ECG monitor.

To monitor events of the heart via an ECG, a series of 3, 5, 6, 10, or 14 or more electrodes may be placed on a patient to sense electrical signals corresponding to activity of a patient's heart. For example, each of the electrodes may be used to allow the charge carriers (electrons) within the electrodes to communicate with the charge carriers (ions) within the body via electrochemical exchange. ECG electrodes on the body surface of a patient allows for voltage changes within the body to be recorded and/or displayed to a heath professional after adequate amplification of the signal.

### SUMMARY

In one aspect, a biomedical connector generally comprises a housing defining an interior space and an opening dimensioned to receive an electrode at least partially into the interior space. A lever is movably attached to the housing and biased toward an engagement position for contacting the electrode when the electrode is received in the opening in the housing to retain the biomedical connector to the electrode. A dampener is disposed in the interior space of the housing and configured to engage the electrode when the electrode is received in the opening in the housing. The dampener is configured to limit movement of the biomedical connector relative to the electrode to reduce motion artifact in a biomedical signal received by the biomedical connector.

In another aspect, a biomedical connector generally comprises a housing defining an interior space and an opening dimensioned to receive an electrode at least partially into the interior space. An electrical contact member is disposed in the interior space of the housing. A foam dampener is disposed in the interior space of the housing and configured to engage the electrode when the electrode is received in the opening in the housing. The foam dampener is configured to limit movement of the biomedical connector relative to the electrode to reduce motion artifact in a biomedical signal received by the biomedical connector.

In yet another aspect, a method of reprocessing a biomedical connector generally comprises deconstructing a housing of the biomedical connector to gain access to an interior space of the housing after the biomedical connector has been placed in use. Removing a dampener from the interior space of the housing. Replacing the removed dampener with a cleaned and sterilized dampener. Reconstructing the housing such that the biomedical connector is in a condition for reuse.

In still another aspect, a biomedical connector generally comprises a housing defining an interior space and an opening dimensioned to receive an electrode at least partially into the interior space. An electrical contact member is disposed in the housing and configured for electrically connecting to the electrode when the electrode is received in the opening in the housing. Foam is disposed in the interior space of the housing, the foam having a rate of recovery of about 10 seconds to about 35 seconds for 100% recovery after deformation.

### BRIEF DESCRIPTION OF THE DRAWINGS

FIG. 1 is an illustration of a patient with a plurality of leads connected to the patient;

FIG. 2 is a perspective of an ECG electrode connector of the present disclosure;

FIG. 3 is a top view of the ECG electrode connector;

FIG. 4 is a side view of the ECG electrode connector;

FIG. 5 is a bottom view of the ECG electrode connector;

FIG. 6 is a top view of the ECG electrode connector with a housing member removed to reveal internal construction;

FIG. 7 is an illustration of a dampener of the ECG electrode connector;

FIG. 8A is a separated view of the ECG electrode connector;

FIG. 8B is a separated view of the ECG electrode connector showing a lever of the connector being actuated;

FIG. 9 is a flow chart of a reprocessing protocol;

FIG. 10 is an illustration of a testing apparatus performing a motion artifact test of the ECG electrode connector;

FIG. 11 is a graph showing motion artifact readings of connectors having different diameters of foam dampeners;

FIG. 12 is a graph showing motion artifact reading for a connector over a period of time.

FIGS. 13A and 13B are graphs comparing motion artifact readings for EGG connectors including foam dampeners of the present disclosure versus ECG connectors without foam dampeners;

FIG. 14A is an illustration comparing ECG signals acquired using EGG connectors including foam dampeners of the present disclosure versus ECG connectors without foam dampeners while subjects perform a marching action;

FIG. 14B is an illustration comparing ECG signals acquired using EGG connectors including foam dampeners of the present disclosure versus ECG connectors without foam dampeners while subjects perform a punching action;

FIG. 15A is an illustration comparing ECG signals acquired using EGG connectors including foam dampeners of the present disclosure versus ECG connectors without foam dampeners while a subject stands on a vibration pad; and

FIG. 15B is another illustration comparing ECG signals acquired using EGG connectors including foam dampeners of the present disclosure versus ECG connectors without foam dampeners while a subject stands on a vibration pad.

Corresponding reference characters indicate corresponding parts throughout the drawings.

### DETAILED DESCRIPTION

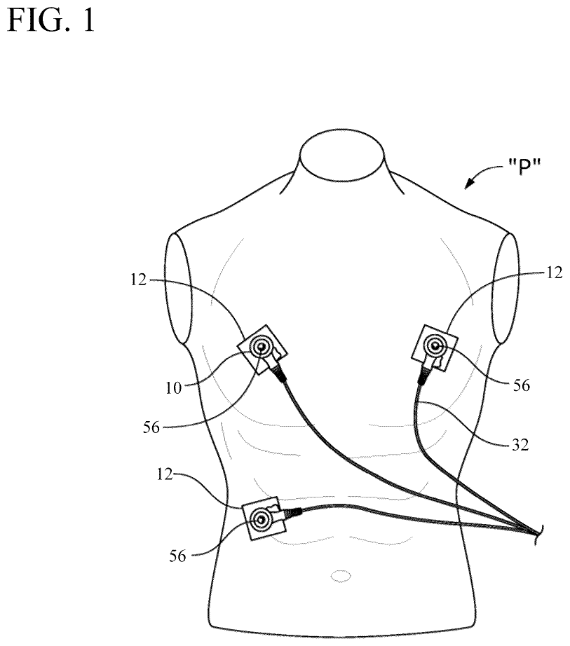

One or more aspects of the present disclosure pertain to biomedical electrode connectors (also interchangeably referred to herein as “patient connectors”) that may be attached to a patient and electronically communicate with a patient monitor via a cable system, in addition to methods of use thereof. Referring to FIG. 1, a set of patient connectors 10 may be connected at various locations on a patient's body to monitor electrical activity of the heart. Movement by the patient can cause forces to be applied to the electrode connector 10 and lead wire extending from the connector. Such motion may result in motion artifact noise in a signal received by the connectors 10. However, by providing the connector features as described herein, the connectors 10 may provide for a reduction in motion artifact resulting from movement of the connectors on the patient. Additionally, processes for reprocessing one or more electrode connectors 10 of the present disclosure are discussed herein. Thus, a hospital or medical facility may commission the connectors 10 for use and then subsequently clean and sanitize one or more components of the connectors, replace spent components, and reconstruct the connector to then permit the recommission of the connectors for use again. Still other aspects of the disclosure are discussed below.

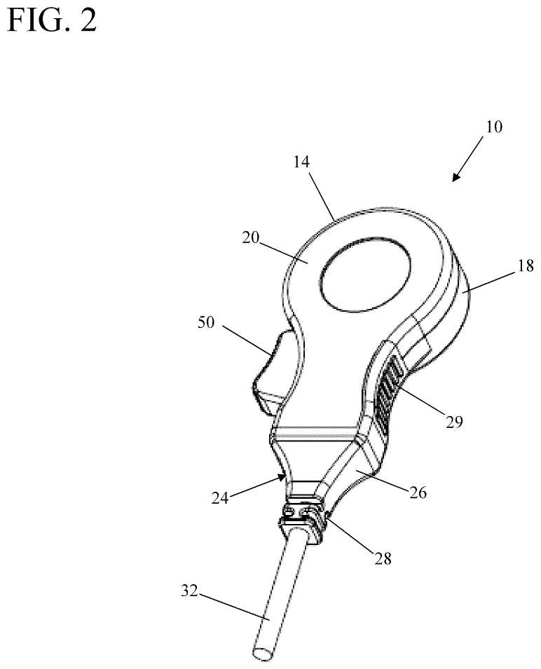

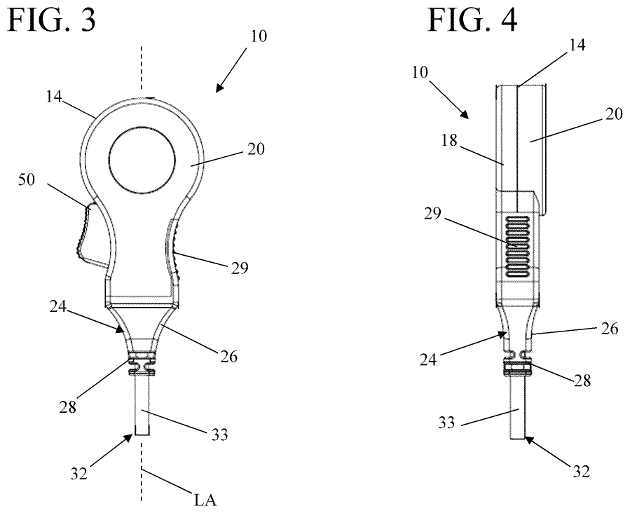

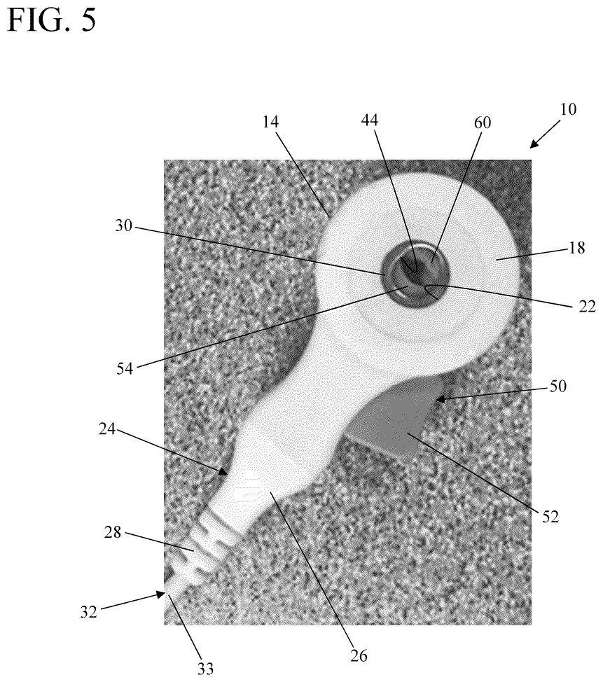

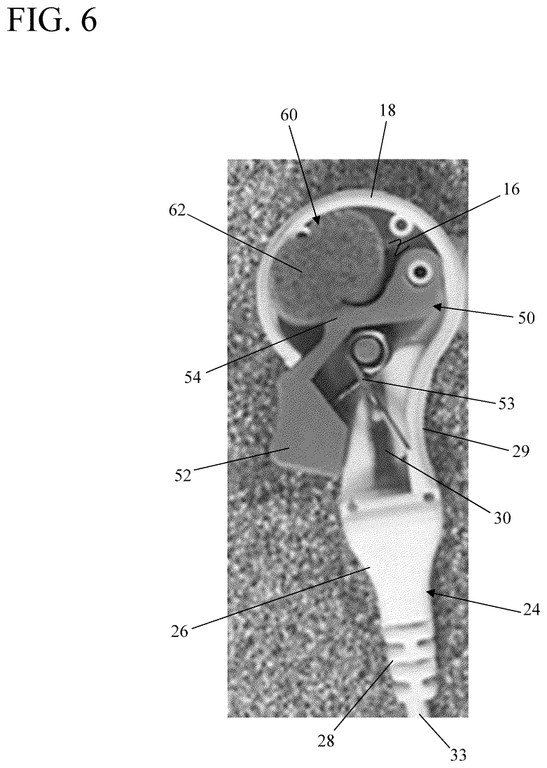

Referring to FIGS. 1-6 generally, each patient connector 10 is configured for connection to an electrode 12 and/or a connection component for use therewith. Electrodes 12 are configured to be attached to a patient's skin in order to measure (or monitor) the electrical activity of the heart and produce a signal that is delivered to a monitoring device. Each connector 10 may include a housing 14 defining an internal cavity 16 (FIG. 6). The housing 14 includes an upper member 18 and a lower member 20 whereby the internal cavity 16 is formed between the upper and lower members. The housing 14 may be formed from a non-conducting material including, without limitation, thermoplastics and/or elastomeric polymer such as polybutylene terephthalate (PBT), polyethylene terephthalate (PET), polyvinyl chloride (PVC), acrylonitrile butadiene styrene (ABS), thermoplastic polyurethanes (TPU), thermoplastic vulcanates (TPV), polypropylene (PP), polyethylene (PE), and/or fiber-reinforced polymer (FRP). In one embodiment, the housing 14 may comprise an injection molded polymer that electrically insulates the patient from the conductive components disposed in the housing. In the illustrated embodiment, the upper and lower members 18, 20 are separate components attached together by suitable means such as by adhesive, ultrasonic welding, or heat welding. The upper housing member 18 defines an opening 22 (FIG. 5) configured to receive a stud 56 (FIG. 1) of the electrode 12. However, the housing 14 could have other configurations without departing from the scope of the disclosure. For example, the housing 14 could be formed by a single housing component.

A strain relief 24 may be attached to the housing 14 and extend generally along (e.g., be centered on) a longitudinal axis LA of the housing. The strain relief 24 includes a neck portion 26 attached directly to a bottom end of the housing 14, and a spine portion 28 disposed at a bottom end of the neck portion. The neck portion 26 and spine portion 28 may be integrally formed. In the illustrated embodiment, the neck portion 26 defines a continuous outer surface that extends from the bottom end of the housing 14 to the bottom end of the neck portion. The outer surface of the neck portion 26 may also taper from a top end to a bottom end such that the top end of the neck portion 26 has a greater cross-sectional dimension than the bottom end of the neck portion. The spine portion 28 may comprise a ribbed body. Thus, the neck portion 26 and the spine portion 28 of the strain relief 24 have different construction. However, both the neck portion 26 and spine portion 28 may be formed from the same material. For example, both the neck portion 26 and spine portion 28 may be formed from a flexible material. In one embodiment, the neck portion 26 and spine portion 28 are formed from a low durometer PVC. Still other materials for the strain relief 24 are envisioned without departing from the scope of the disclosure.

A grip portion 29 extends upward from the neck portion 26 of the strain relief 24 and is disposed on a side of the connector housing 14 opposite a lever 50. Thus, the grip portion 29 is configured to be engaged by a user's finger (e.g., the index finger) when the user's thumb is on the lever 50. The grip portion 29 may include a plurality of ribs and/or recesses on an exterior surface of the grip portion to provide traction for the user's finger. The grip portion 29 may be comprised of the same material as the strain relief 24. In one embodiment, the strain relief 24 and grip portion 29 are integrally formed as one piece of material.

Referring to FIGS. 5 and 6, a contact member 30 may also be supported in the housing 14 and electrically connected to a lead wire 32 that is electrically connected to a monitoring device (e.g., ECG monitor). For example, the contact member 30 may be connected to the lead wire 32 by crimping, soldering, or welding. The contact member 30 may be constructed from any suitable electrically conductive material, including without limitation stainless steel or low-carbon steel. Alternatively, the contact member 30 could be constructed from a non-conductive material having a conductive coating. The lead wire 32 may include an external portion (i.e. shielded wire 33 extending away from the connector) and an internal portion (i.e., portion of the lead wire disposed inside the connector). The lead wire 32 may be supported at its exit location from the housing 14 by the strain relief 24. As will be understood, the strain relief 24 is configured to improve a wire termination breakage force of the lead wire 32 by reducing tension/strain on the wire.

The contact member 30 defines a contact opening 44 that is in communication with the internal cavity 16 of the housing 14. A lever 50 may be pivotably connected to the housing 14. In the illustrated embodiment, the lever 50 includes an actuating end 52 and may be biased to a first (engagement) position by a biasing member 53 (FIG. 6). The lever 50 may include an engaging region 54 extending from the actuating end 52 so as to project across a portion of the contact opening 44 when the lever is in the first position. In use, lever 50 is actuatable to a second (receiving) position wherein the engaging region 54 does not obstruct or extend across the portion of the contact opening 44. For example, a clinician may apply pressure to the actuating end 52 that is sufficient to overcome the biasing force of the biasing member 53, thereby causing the engaging region 54 to move to the second position. In this position, the stud of the electrode 12 may be received through the opening 22 in the housing 14 and into the contract opening 44.

Referring to FIG. 1, the connector 10 may be adapted for connection to a conventional biomedical electrode assembly 12 (e.g., an ECG electrode assembly). A typical ECG electrode assembly may include an electrically conductive layer and a backing layer (broadly, a base). The electrode assembly 12 has a patient contact side and a connector side. The contact side of the electrode may include biocompatible conductive gel or adhesive for affixing the electrode to a patient's body for facilitating an appropriate electrical connection between a patient's body and the electrode assembly. The connector side of the electrode may incorporate a metallic press stud 56 extending in transverse relation to the base having a bulbous profile whereby an upper portion has a greater cross-sectional dimension than a lower portion for coupling the electrode to the connector 10. In use, the clinician removes a protective covering from the contact side to expose the gel or adhesive, affixes the electrode to the patient's body, and attaches the appropriate ECG lead wire connector (e.g., connector 10) to the press stud 56. Connection of the connector 10 to the electrode occurs when the lever 50 is actuated to allow the contact opening 44 to receive the press stud 56, and then released so that the biasing member 53 moves the engaging region 54 of the lever against the head of the male press stud 56 to retain the stud in the contact opening. The biasing force of biasing member 53 helps to maintain the press stud 56 within the contact opening 44 and in engagement with the contact plate 30. Thus, the biased lever 50 inhibits removal or electrical disconnection of the electrode from ECG connector 10.

Referring to FIGS. 5-7, a dampener 60 is disposed within the internal cavity 16 of the housing 14. The dampener 60 is configured to engage the stud 56 of the electrode 12 when the stud is received through the opening 22 in the housing 14 and into the contact opening 44 to secure the connector 10 to the electrode. The dampener 60 is configured to limit movement of the connector 10 relative to the electrode 12 to reduce motion artifact in a biomedical signal received by the connector. As such, the dampener 60 may comprise a resilient, compressible material such that the dampener is compressed upon engagement with the stud 56. The compression of the dampener 60 may cause the dampener to at least partially surround lateral sides of the stud 56. This engagement of the dampener 60 with the stud 56 helps to stabilize the connector 10 on the electrode 12. In one embodiment, the dampener 60 surround at least half of an outer circumference of the stud 56.

In the illustrated embodiment, the dampener 60 is disposed in the open area in the upper member 18 opposite the lever 50. As such, the dampener 60 is allowed to freely expand into the open area in the upper member 18. However, the dampener 60 could be disposed within a subdivided section (i.e., internal wall) within the upper member 18 to limit the expansion of the dampener 60. In this embodiment, the compression of the dampener 60 can be controlled to apply a specific amount of compliance in response to engagement by the stud 56. The subdivided section may also provide an indication to the manufacturer of where to locate the dampener 60 in the housing 14.

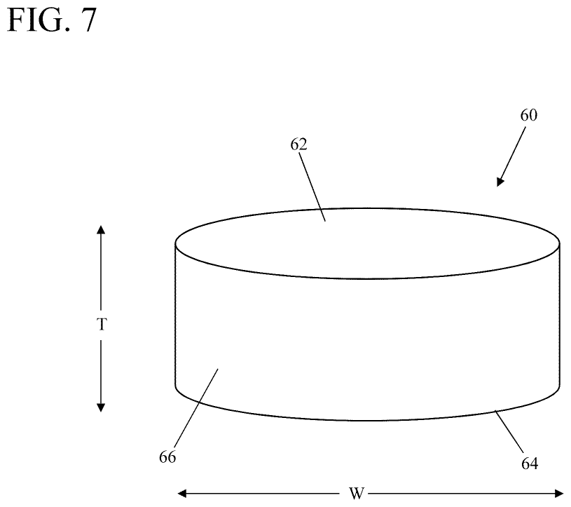

The dampener 60 may comprise a cylindrical, disc-shaped member having a top surface 62, a bottom surface 64, and a circumferentially extending side surface 66 extending between the top and bottom surfaces (FIG. 7). A thickness T of the dampener 60 extends from the top surface 62 to the bottom surface 64. In one embodiment, the thickness T is about ¼ inch. A width W of the dampener 60 extends laterally across the dampener. In the illustrated embodiment, the dampener 60 is cylindrical. Thus, a diameter of the dampener 60 defines the width W of the dampener. In one embodiment, the width W of the dampener 60 is less than about ½ inch. In one embodiment, the width W of the dampener 60 is between about 0.2 inches and about 0.4 inches. The thickness T and width W of the dampener 60 are measured with the dampener is in an undeformed state such that the shape of the dampener is not altered as a result of its interaction with the housing 14 or any other components of the connector 10. Therefore, the thickness T and width W measurements are taken when the dampener 60 is removed from the housing 14 and in its natural undeformed state. It will be understood that the dampener 60 may have other dimensions and configurations without departing from the scope of the disclosure. For example, the dampener 60 may have a semi-circle, oval, or crescent shape.

The dampener may be formed from any suitable material. The material of the dampener 60 may be selected for its resiliently yieldable nature so that the dampener at least partially deforms upon engagement by the electrode 12. However, the material may also be selected based on the material's ability to provide some resistance or counterforce in response to the material being deformed to stabilize the connector 10 on the electrode 12. Thus, the material may be selected based on a range of softness that allows for both deformation and stability. In one embodiment, the dampener 60 comprises a foam material. In particular, the dampener 60 may comprise a viscoelastic foam. One example of a suitable material is Xodus Pink Pad Memory Foam available from Xodus Medical Inc. of New Kensington, Pennsylvania.

Such a foam material may be selected based on its density. For example, a density of the foam material may be indicative of the ability of the foam to both cushion and stabilize the electrode 12. In one embodiment, the dampener material has a density of between about 80 kg/m³ and about 300 kg/m³. In one embodiment, the dampener material has a density of between about 80 kg/m³ and about 105 kg/m³. In one embodiment, the dampener material has a density of about 225 kg/m³. Still other densities and material properties may be considered in the selection of the dampener. In one embodiment, the dampener 60 is electrically conductive.

For example, a rate of recovery may also be considered in the selection of the dampener 60. The rate of recovery pertains to the time required for a viscoelastic foam to return to its starting shape after the foam has been deformed. In one embodiment, the rate of recovery of the dampener 60 is in the range of approximately 2-35 seconds for approximately 50% to 100% recovery after deformation. In one embodiment, the rate of recovery of the dampener 60 is in the range of approximately 2-10 seconds for approximately 50% to 80% recovery after deformation. In one embodiment, the rate of recovery of the dampener 60 is in the range of approximately 10-15 seconds for approximately 80% to 100% recovery after deformation. In one embodiment, the rate of recovery is in the range of approximately 6-15 seconds for approximately 80% to 90% recovery after deformation. In one embodiment, the rate of recovery is in the range of approximately 10-35 seconds for 100% recovery after deformation.

Testing method ASTM D3574-17 Test M: Recovery Time was used on the foam material to measure recovery time. In particular, foam samples were stacked on top of each other to reach a target thickness for testing. For example, a thickness of 100 mm was used for the sample thickness. An apparatus having a flat circular indenter foot was connected to a crosshead of a universal test frame by a swivel joint. The circular indenter had a diameter of about 200 mm. A base plate with approximately 6.5 mm holes spaced on 20 mm centers was used to support the sample stack. The sample stack was preflexed by 75% of its thickness, twice, at a rate of 250 mm/min. The crosshead was then withdrawn and the sample stack was allowed to rest with no contact for 6 min. The indenter foot was then brought into contact with the sample stack at a rate of 50 mm/min until a contact force of 4.5N was achieved. The thickness of the sample stack at this force was recorded as the contact force thickness. The sample stack was then compressed by 75% of the contact force thickness at a rate of 1000 mm/min and then allowed to rest for 1 min. at that compression. Next, the indenter foot was withdrawn to a position where the sample stack would be compressed by 5% of the contact force thickness. The time between the withdrawal of the indenter foot and the return of 4.5N was recorded as the recovery time.

Additionally, other types of materials may be used for the dampener 60. For example, the foam may comprise other types of memory foam, carpenter foam, or spray foam. Still other types of foam are envisioned without departing from the scope of the disclosure.

Referring to FIGS. 5 and 6, when the dampener 60 is disposed within the internal cavity 16 of the housing 14, a portion of the dampener extends across the opening 22 in the housing such that a portion of the dampener is registration with the opening. The dampener 60 also occupies a space in the housing opposite the engaging region 54 of the lever 50. In particular, the dampener 60 generally opposes the engaging region 54 of the lever 50 about the opening 22 in the housing 14. In the illustrated embodiment, the engaging region 54 generally circumscribes a first half of the opening 22, and the dampener 60 generally circumscribes a second half of the opening. Therefore, when the stud 56 of the electrode 12 is received in the opening 22 of the housing 14, the dampener 60 extends around about half of the circumference of the stud. This allows the dampener to stabilize the connector 10 on the electrode 12 to reduce the relative movement between the connector and electrode. As a result, the dampener 60 is able to reduce the motion artifact in the biomedical signal received by the connector 10 to provide a clearer signal with less noise to the monitoring device connected to the connector.

Additionally, the dampener 60 may be sized and/or positioned such that the dampener does not undesirably obstruct the stud 56 from being received in the electrode 12. In one embodiment, the engaging region 54 is configured to engage the dampener 60 when the lever 50 is actuated to the receiving position to move the dampener away from the opening 22 in the housing 14 to allow the stud to be received in the housing. Additionally or alternatively, the dampener 60 may be shaped such that the portion in registration with the opening 22 in the housing 14 is configured to accommodate the shape of the stud 56 to allow the stud to be received in the housing.

In the illustrated embodiment, a lever-type connector 10 is disclosed. However, the dampener 60 may be provided in other connector types without departing from the scope of the disclosure. For example, jaw type connectors, push button connectors, and “wire out of top” connectors may also incorporate the dampener 60 to address motion artifact.

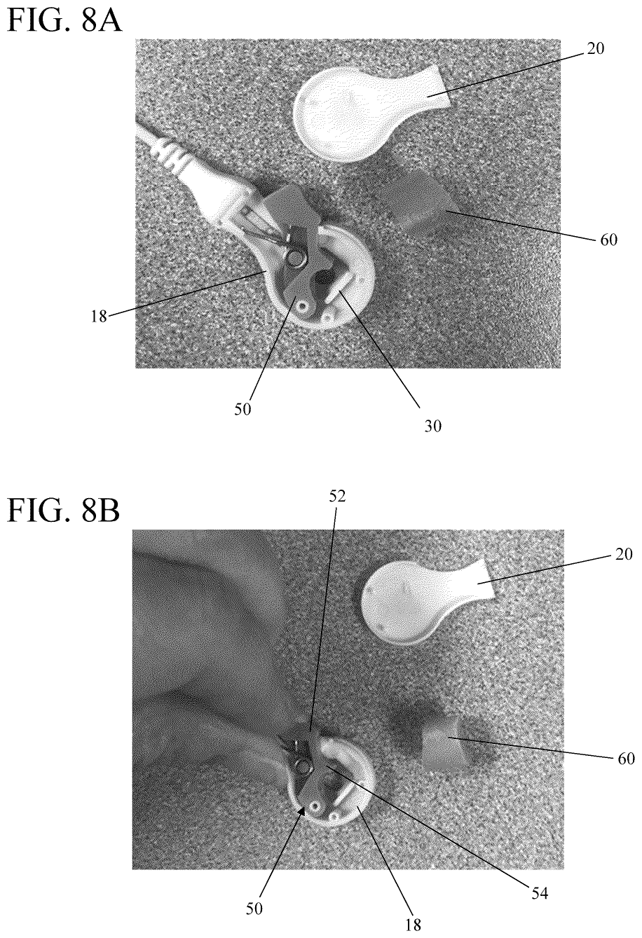

Referring to FIGS. 8A and 8B, a process for installing the dampener 60 in the connector 10 is shown. First, the lower member 20 is separated from the upper member 18 of the housing 14 to allow for access to the internal cavity 16 of the upper member (FIG. 8A). It will be understood, that the installation of the dampener 60 may also occur prior to attaching the upper member 18 to the lower member 20. The actuating end 52 of the lever 50 is then actuated to pivot the engaging region 54 of the lever away from the open space in the upper member 18 (FIG. 8B). The dampener 60 can then be placed inside the interior space of the upper member 18. The lever 50 is then be released. The engaging region 54 of the lever 50 may engage the dampener 60 to press the dampener against the side of the upper member 18 to at least temporarily hold the dampener in the upper member (FIG. 6). The lower member 20 is then attached to the upper member 18 to retain the dampener 60 in the housing 14. Thus, in the illustrated embodiment, the dampener 60 is retained in the housing 14 without using any additional attachment means such as adhesive. Alternatively, the dampener 60 may be affixed in the housing 14 using an adhesive or other suitable connection to the housing. In the embodiment where the dampener 60 comprises spray foam, the dampener may be sprayed into the housing 14 without detaching the upper and lower members 18, 20.

Additionally, the connector 10 may be reprocessed after the connector has been used in a clinical setting to clean and sanitize one or more components of the connector so that the connector can again be used in the field. Generally, reprocessing may include method steps such as inspecting, cleaning, disinfecting or sanitizing, high level disinfecting, and/or testing. Further, the term reconstructing may include method steps other than inspecting, cleaning, disinfecting or sanitizing, high level disinfection, and/or testing, such as repair of connectors, wires, and/or conduits, replacing a cover, and/or replacing other components of the connector. In one example, an apparatus subjected to reconstruction may be subject to additional processing or repair beyond reprocessing. However, the aforementioned terms and definitions are merely provided as examples. Thus, the reprocessing, reconstructing, and/or refurbishment process in combination with the various other features of the connector 10 described herein may provide for a reduction in waste and/or cost by allowing for efficient reprocessing, reconstructing, and/or refurbishment of the connector and/or lead set.

Additionally, the dampener 60 may be consumable due to contamination by liquids and/or particulates that cannot be fully removed by conventional reprocessing methods. Therefore, the dampener 60 can be a disposable component that can to be replaced during reprocessing requiring disassembly of the housing 14.

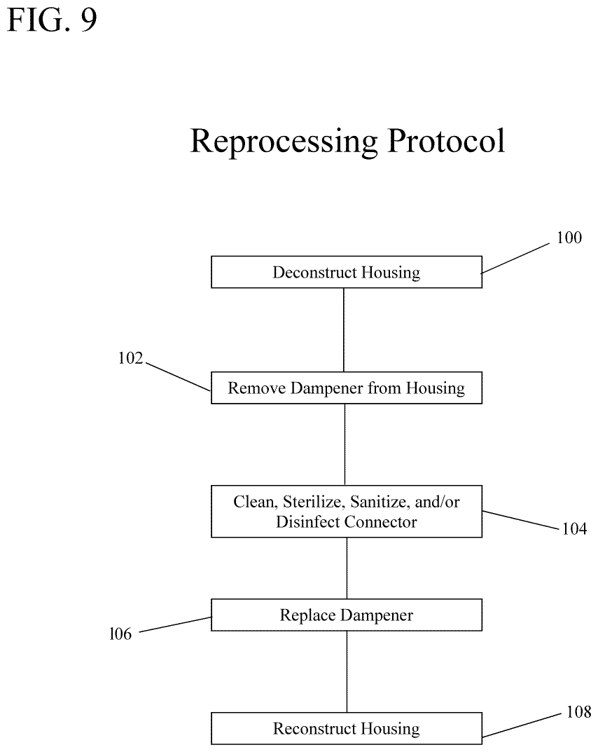

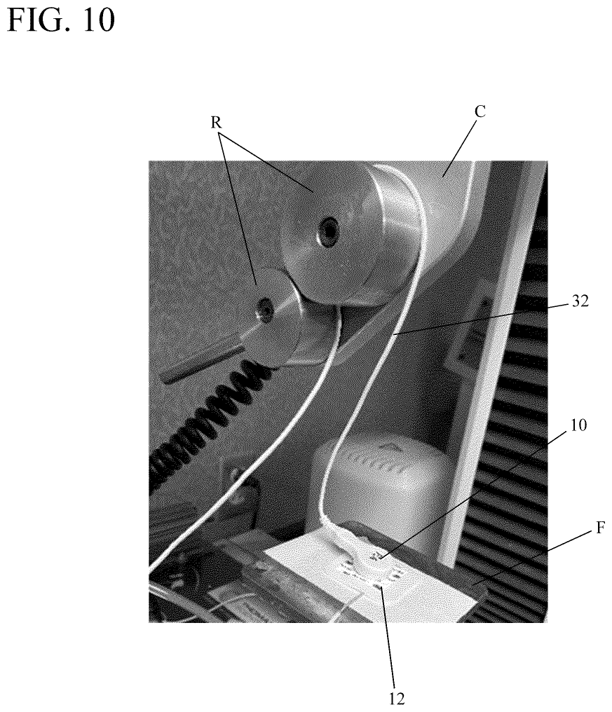

Referring to FIG. 9, the connector 10 may be reprocessed according to a reprocessing protocol. In various embodiments, the connector 10 is deconstructed to allow access to the internal cavity 16 of the housing 14 at 100. In particular, the upper member 18 of the housing 14 may be detached from the lower member 20. The dampener 60 can then be removed from the housing 14 at 102. One or more components of the connector 10 can then be cleaned, sterilized, sanitized, and/or high level disinfected at 104. This may include cleaning the dampener 60 that was removed from the housing 14 or providing a new dampener in such cases where cleaning, sterilizing, etcetera is not viable. Additional components of the connector 10 may also be cleaned during the reprocessing. The cleaned or new dampener 60 is then located in the housing 14 at 106. For example, the dampener 60 is placed in the housing 14 generally opposite the engaging region 54 of the lever 50. Once all the intended components of the connector 10 are cleaned, the housing 14 is reconstructed to place the connector back in condition for use at 108. In particular, the upper member 18 may be reattached to the lower member 20 to again retain the dampener 60 in position in the housing 14. Therefore, the reprocessing of the connector 10 includes both cleaning and reconfiguring the connector after the connector has been placed in clinical use. In the embodiment where spray foam is used for the dampener 60, the dampener may be dissolved as part of the reprocessing process and replaced with new spray foam.

Referring to FIGS. 10-12, a study was conducted using connectors including dampeners as disclosed in the present disclosure to measure the effects of the dampeners on the motion artifact readings when the connectors were subjected to a pulling force when attached to an electrode. During one phase of the study, four (4) different test groups where tested. Each test group consisted of five (5) connectors. The connectors in each test group including a foam dampener for reducing motion artifact. The foam dampener consisted of the Xodus Pink Pad Memory Foam. All the foam dampeners used in the test were cylindrical discs sized to have an approximately 1-inch thickness. However, each test group included dampeners having different diameters. The connectors in a first test group included foam dampeners having diameters of 5/16 inches. The connectors in a second test group included foam dampeners having diameters of ⅜ inches. The connectors in a third test group included foam dampeners having diameters of 7/16 inches. The connectors in a fourth test group included foam dampeners having diameters of ½ inches.

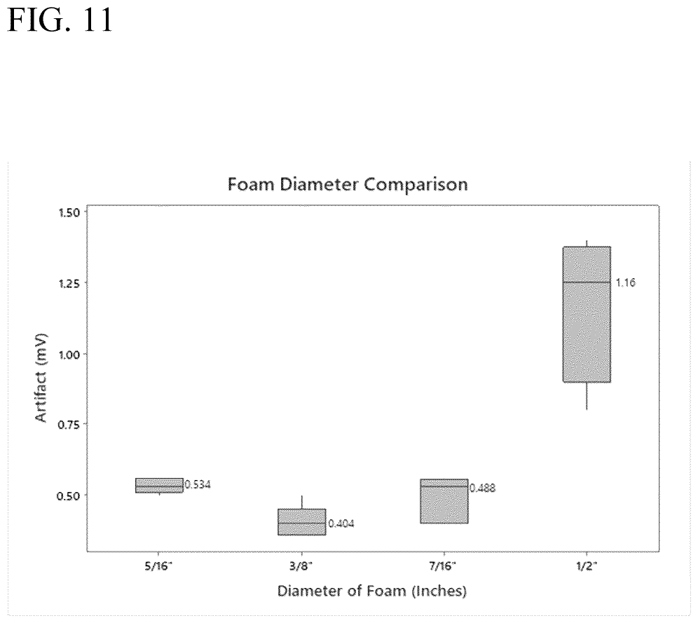

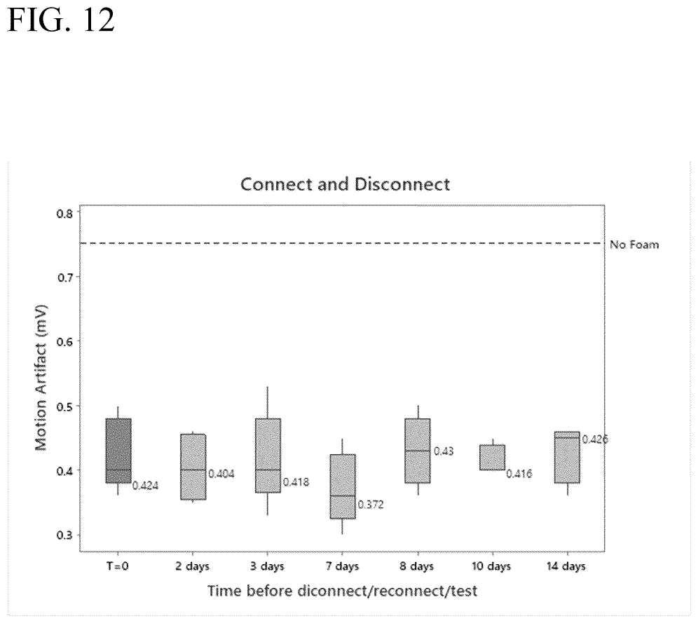

For the motion artifact readings, the connectors were attached to an electrode mounted on a lower fixture F (FIG. 10). The lead wires 32 of the connectors were wrapped around roller clamps R on an Instron Model 3365 machine (FIG. 10). The roller clamps R are mounted on a moveable crosshead C. The Instron machine was then operated to exert a pulling force on the lead wire 32 by moving the crosshead C upwards away from the lower fixture. This movement preloads the tension in the lead wire 32 to 0.1 lbf. The crosshead C was then oscillated a distance of 10 mm. at a rate of 40 in/min for 6 cycles. An electric circuit and patient monitor were used to measure the motion artifact created from the force exerted by the Instron. FIG. 11 shows a graph comparing the motion artifact readings from each of the four groups. As can be seen, the first three groups were particularly successful in reducing the motion artifact with each group having a recorded motion artifact value of less than 0.6 mV. The second group having dampener diameters of ⅜ inches performed the best at reducing motion artifact with readings of about 0.4 mV. By comparison, motion artifact values for connectors without a dampener have been found to exceed 0.7 mV (FIG. 12).

Referring to FIG. 12, another phase of the study monitored the performance of the ⅜-inch dampener over time. A group of five (5) connectors were tested. During this test, the connectors were attached to the electrodes for a period of fourteen (14) days. Prior to taking a motion artifact reading, the connectors 10 were disconnected from the electrodes 12 and allowed to sit for two (2) minutes. The connectors 10 were then reattached to the electrodes 12 and the motion artifact readings were taken. This process of connecting and disconnected the connectors 10 was used to simulate the use of the connectors in the clinical environment. The test concluded that, the dampeners were successful in reducing motion artifact through the entire duration of the test. In particular, the averages of the motion artifact readings remained between about 0.37 mV and about 0.43 mV. By comparison, a control connector without a dampener was studied over the 14 days. The motion artifact readings for the control connector remained constant at about 0.75 mV as indicated by the dotted line labeled “No Foam”. Therefore, the study concluded that the connectors incorporating dampeners are viable as a solution for reducing motion artifact in a clinical setting.

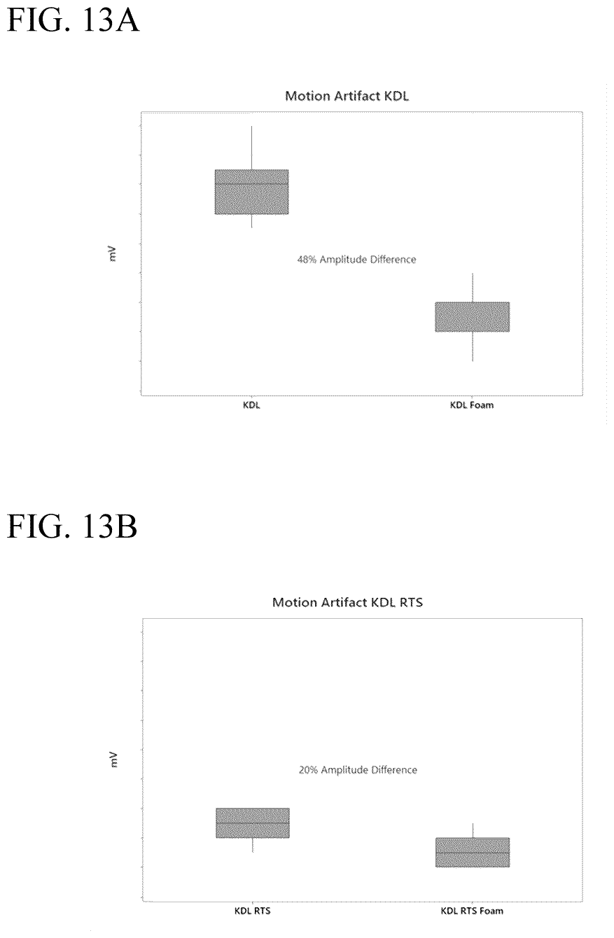

Referring to FIGS. 13A and 13B, another study was conducted comparing connectors including dampeners as disclosed in the present disclosure to connectors without dampeners to measure the effects of the dampeners on the motion artifact readings when the connectors were subjected to a pulling force when attached to an electrode as previously described. FIG. 13A compares the Cardinal Health™ Kendall DL™ (KDL) ECG Leads with a dampener to the Cardinal Health™ Kendall DL™ (KDL) ECG Leads without a dampener. Notably, the KDL Leads include metal electrical contact member within the housings. As can be seen, the motion artifact readings for the KDL Leads with a foam dampener were about 48% lower than the motion artifact readings for the KDL Leads without a foam dampener. Similarly, FIG. 13B compares the Cardinal Health™ Kendal DL™ RTS (KDL RTS) Leads with a dampener to the Cardinal Health™ Kendal DL™ RTS without a dampener. In comparison, the KDL RTS Leads include non-metal electrical contact member within the housings. As can be seen, the motion artifact readings for the KDL RTS Leads with a foam dampener were about 20% lower than the motion artifact readings for the KDL RTS Leads without a foam dampener.

The increased reduction in motion artifact between the KDL Leads and the KDL RTS Leads can be attributed to the metal to metal contact created by the metal electrical contact member of the KDL Leads engaging the electrode when the connector is attached to the electrode. Therefore, the metal-to-metal contact results in greater motion artifact readings for the KDL Leads without dampeners as comparted to the KDL RTS Leads without dampeners. However, the addition of the foam dampeners was able to reduce the motion artifact readings for both connectors to around the same amount. Thus, the additional study confirmed the ability of the dampeners to significantly reduce motion artifact in different types of ECG connectors.

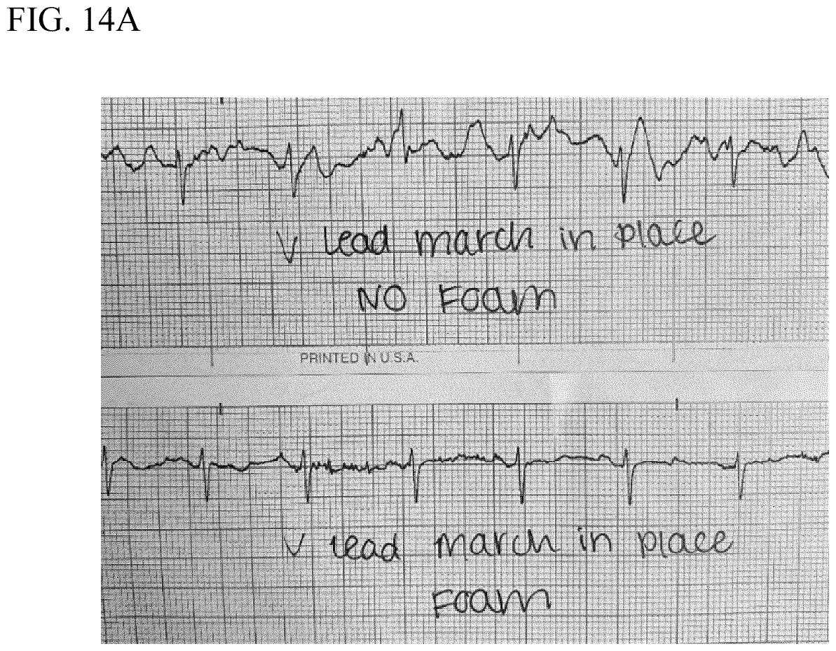

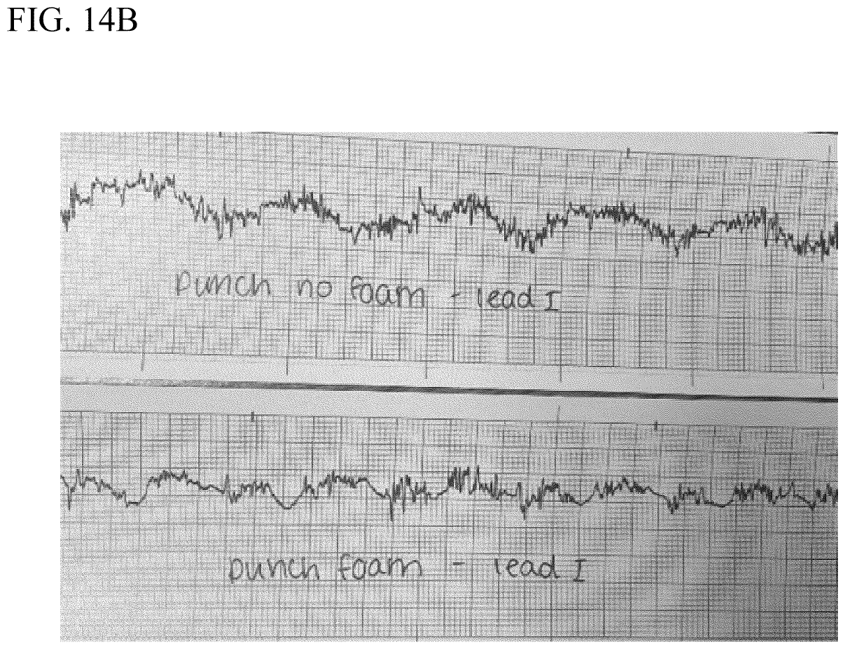

Referring to FIGS. 14A and 14B, exercise testing was conducted on the Cardinal Health™ Kendal DL™ (KDL) Leads in comparing the KDL Leads with foam dampeners as disclosed in the present disclosure with KDL Leads without foam dampeners. A first exercise study (FIG. 14A) was conducted in which a user having a 5-lead KDL Lead Set attached to their person for obtaining an ECG reading performed a marching action (i.e., marched in place). The ECG readings were recorded during the marching and a comparison was made between the KDL Leads with a foam dampener and the KDL Leads without a dampener. As is shown in FIG. 14A, the inclusion of the foam dampener greatly reduced the noise in the ECG signal providing clearer output readings while the user performed the marching action.

Similarly, a second exercise study (FIG. 14B) was conducted in which a user having a 5-lead KDL Lead Set attached to their person for obtaining an ECG reading performed a punching action. The ECG readings were recorded during the punching and a comparison was made between the KDL Leads with a foam dampener and the KDL Leads without a dampener. As is shown in FIG. 14B, the inclusion of the foam dampener greatly reduced the noise in the ECG signal providing clearer output readings while the user performed the punching action.

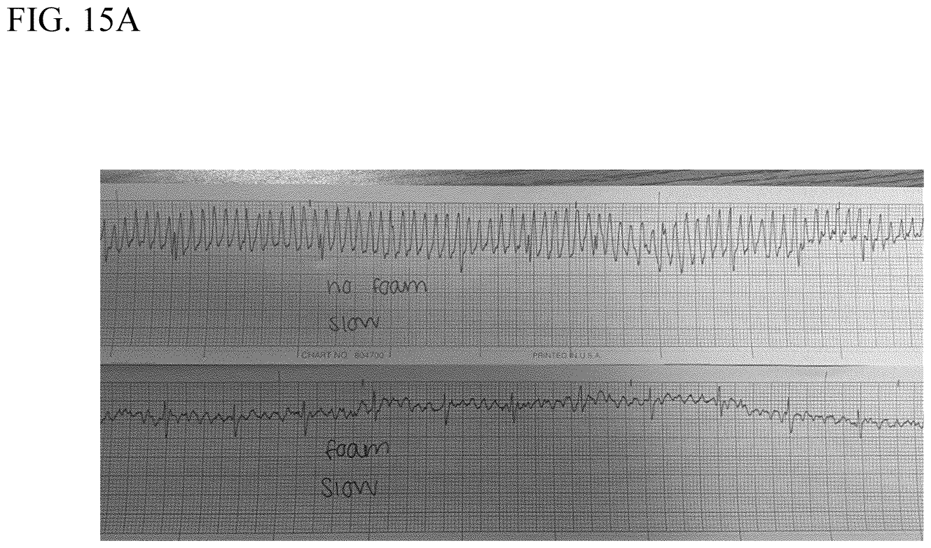

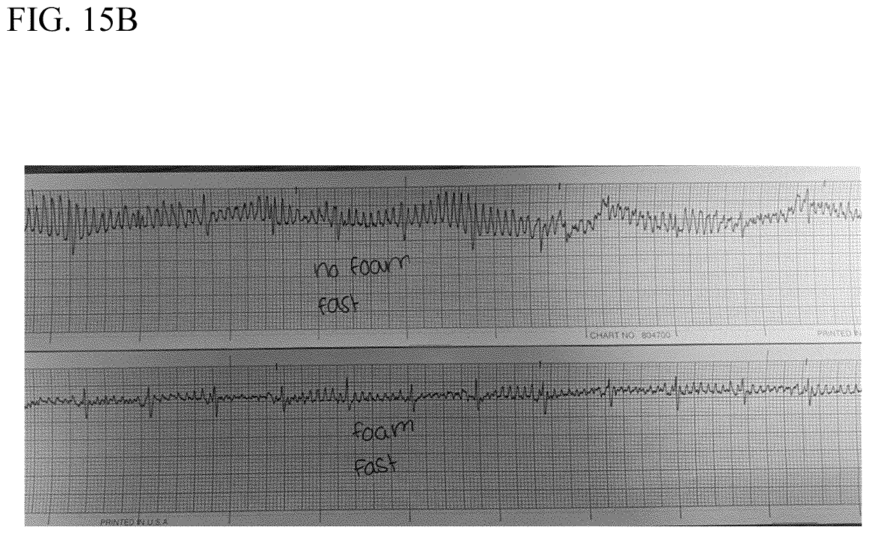

Referring to FIGS. 15A and 15B, vibration testing was conducted on the Cardinal Health™ Kendal DL™ (KDL) Leads in comparing the KDL Leads with foam dampeners as disclosed in the present disclosure with KDL Leads without foam dampeners. A first vibration study (FIG. 15A) was conducted in which a user having a 5-lead KDL Lead Set attached to their person for obtaining an ECG reading stood on a vibration pad that vibrated at a “slow” rate. The ECG readings were recorded during the vibration and a comparison was made between the KDL Leads with a foam dampener and the KDL Leads without a dampener. As is shown in FIG. 15A, the inclusion of the foam dampener greatly reduced the noise in the ECG signal providing a clearer output reading.

A second vibration study (FIG. 15B) was conducted in which a user having a 5-lead KDL Lead Set attached to their person for obtaining an ECG reading stood on a vibration pad that vibrated at a “fast” rate. The ECG readings were recorded during the vibration and a comparison was made between the KDL Leads with a foam dampener and the KDL Leads without a dampener. As is shown in FIG. 15B, the inclusion of the foam dampener greatly reduced the noise in the ECG signal providing a clearer output reading.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions without departing from the scope of the invention, it is intended that all matter contained in the above description and shown in the accompanying drawings shall be interpreted as illustrative and not in a limiting sense.

What is claimed is:

1. A biomedical connector comprising:
a housing defining an interior space and an opening dimensioned to receive an electrode at least partially into the interior space;
a lever movably attached to the housing and biased toward an engagement position for contacting the electrode when the electrode is received in the opening in the housing to retain the biomedical connector to the electrode; and
a dampener disposed in the interior space of the housing and configured to engage the electrode when the electrode is received in the opening in the housing, the dampener being configured to limit movement of the biomedical connector relative to the electrode to reduce motion artifact in a biomedical signal received by the biomedical connector.

2. The connector of claim 1, wherein the dampener comprises foam.

3. The connector of claim 2, wherein dampener comprise a viscoelastic foam.

4. The connector of claim 2, wherein the dampener has a thickness extending between top and bottom surfaces of the dampener and a width extending laterally across the dampener, a maximum width of the dampener being less than about ½ inch when the dampener is in an undeformed state.

5. The connector of claim 4, wherein the maximum width of the dampener is between about 0.3 inches and about 0.4 inches when the dampener is in the undeformed state.

6. The connector of claim 2, wherein the foam has a density of between about 80 kg/m³ and about 300 kg/m³.

7. The connector of claim 1, wherein at least a portion of the lever and dampener are disposed at opposite sides of the opening in the housing such that the lever and dampener are configured to engage the electrode on diametrically opposite sides of the electrode.

8. The connector of claim 1, wherein the dampener is configured to deform upon engagement by the electrode to at least partially surround lateral sides of the electrode.

9. The connector of claim 8, wherein the dampener is configured to surround at least half of an outer circumference of the electrode.

10. The connector of claim 1, wherein the dampener extends partially across the opening in the housing prior to receiving the electrode in the opening.

11. The connector of claim 1, wherein the connector comprises reprocessed components.

12. The connector of claim 1, further comprising an electrical contact member disposed in the housing.

13. The connector of claim 12, further comprising a lead wire disposed in the housing, wherein an end of the lead wire is electrically coupled to the electrical contact member.

14. The connector of claim 13, wherein the electrical contact member defines an opening, the lever being biased to the engagement position by a biasing member, the lever including an actuating end and an engaging region, the lever extending across a portion of the contact opening when the lever is in the engagement position.

15. The connector of claim 14, wherein the lever is actuatable against the bias of the biasing member to a receiving position wherein the engaging region does not obstruct or extend across the portion of the contact opening permitting the electrode to be received in the opening in the housing.

16. The connector of claim 1, wherein a motion artifact value of the connector is less than 0.6 mV.

17. The connector of claim 1, wherein a motion artifact value of the connector is maintained between about 0.3 mV and about 0.5 mV during use of the connector over 14 days.

18. The connector of claim 1, wherein the dampener is electrically conductive.

19. A biomedical connector comprising:
a housing defining an interior space and an opening dimensioned to receive an electrode at least partially into the interior space;
an electrical contact member disposed in the interior space of the housing; and
a foam dampener disposed in the interior space of the housing and configured to engage the electrode when the electrode is received in the opening in the housing, the foam dampener being configured to limit movement of the biomedical connector relative to the electrode to reduce motion artifact in a biomedical signal received by the biomedical connector.

20. The connector of claim 19, wherein the electrical contact member comprises metal.

21. The connector of claim 19, wherein the foam dampener has a density of between about 80 kg/m³ and about 300 kg/m³.

22. The connector of claim 19, wherein the foam dampener comprises a viscoelastic foam.

23. The connector of claim 19, wherein the foam dampener has a thickness extending between top and bottom surfaces of the foam dampener and a width extending laterally across the foam dampener, a maximum width of the foam dampener being less than about ½ inch when the foam dampener is in an undeformed state.

24. A method of reprocessing a biomedical connector comprising:
deconstructing a housing of the biomedical connector to gain access to an interior space of the housing after the biomedical connector has been placed in use;
removing a dampener from the interior space of the housing;
replacing the removed dampener with a cleaned and sterilized dampener; and
reconstructing the housing such that the biomedical connector is in a condition for reuse.

25. The method of claim 24, wherein replacing the removed dampener comprises one of cleaning and sterilizing the removed dampener or providing a cleaned and sterilized dampener different from the removed dampener.

26. The method of claim 25, wherein deconstructing the housing comprises separating an upper housing member from a lower housing member.

27. The method of claim 26, wherein reconstructing the housing comprises reattaching the upper housing member to the lower housing member.

28. The method of claim 25, wherein replacing the removed dampener comprises placing the cleaned and sterilized dampener in the housing generally opposite an engagement portion of a lever pivotally mounted on the housing.

29. A biomedical connector comprising:
a housing defining an interior space and an opening dimensioned to receive an electrode at least partially into the interior space;
an electrical contact member disposed in the housing and configured for electrically connecting to the electrode when the electrode is received in the opening in the housing; and
foam disposed in the interior space of the housing, the foam having a rate of recovery of about 10 seconds to about 35 seconds for 100% recovery after deformation.

30. The connector of claim 29, wherein the rate of recovery is about 10 seconds to about 15 seconds for 100% recovery after deformation.

31. The connector of claim 29, wherein the foam has a density of between about 80 kg/m³ and about 300 kg/m³.

32. The connector of claim 29, wherein the foam comprises a viscoelastic foam.

33. The connector of claim 29, wherein the foam has a thickness extending between top and bottom surfaces of the foam and a width extending laterally across the foam, a maximum width of the foam being less than about ½ inch when the foam is in an undeformed state.

34. The connector of claim 33, wherein the maximum width of the foam is between about 0.3 inches and about 0.4 inches when the foam is in the undeformed state.

35. The connector of claim 29, wherein the foam is configured to engage the electrode when the electrode is received in the opening in the housing.
